(19) United States
(12) Patent Application Publication
(10) Pub. No.: US 2002/0098110 A1
(43) Pub. Date: Jul. 25, 2002

(54) Exterior panels containing algae-inhibiting properties

(76) Inventors: William David Graham, Granville, OH (US); Daniel Nelson Leavell, Granville, OH (US); David George Miller, Pickerington, OH (US); Margaret Mary Woodside, Pickerington, OH (US); Carla Ann Miller, Newark, OH (US); David Paul Aschenbeck, Newark, OH (US); James Spray Belt, Utica, OH (US)

Correspondence Address: OWENS CORNING, 2790 COLUMBUS ROAD, GRANVILLE, OH, 43023, US

(21) Appl. No.: 09/727,910

(22) Filed: Nov. 30, 2000

(51) Int. Cl. A61L 2/23

(52) U.S. Cl. 422/28; 424/413

(57) ABSTRACT

An exterior panel containing algaecidal properties is provided for inhibiting the growth of algae on roofing materials and exterior siding. Algaecide is applied to the exterior panel on the unexposed surface, i.e., the side of the panel, when installed, is not directly in contact with the effects of weather. Moisture such as rain, dew, melting frost or snow, contacts the exposed surface of the panel and water is transported to the bottom portion of the unexposed surface of the panel via capillary attraction. Water extracts the algaecide and distributes it to the exposed surfaces of the panels and all of the other descending panels.

### TECHNICAL FIELD AND INDUSTRIAL APPLICABILITY OF THE INVENTION

The present invention relates to controlling algae growth on building materials. More particularly, it relates to a controlled release of algae control materials onto the exposed surfaces of exterior panels, including roofing materials.

### BACKGROUND OF THE INVENTION

Controlling the growth of algae and fungi on roofing materials is well known in the art. In the past, the exposed surfaces of roofing shingles have been treated with algaecide to inhibit algae and fungi growth. A problem with coating the exposed surfaces is that the algaecide is gradually stripped away by rain and other inclement weather. As a result, the algaecidal properties of the shingle are significantly reduced. For example, U.S. Pat. No. 3,598,627 illustrates embedding the weathering surface of roofing and siding shingles with zinc or zinc-containing materials to prevent the growth of algae and fungi.

U.S. Pat. No. 5,599,586 shows coating the exterior surface of a cement tile or composite asphalt product with a thermoplastic film containing organo-metallic complexes to protect the exterior surface against the growth of algae. Rainwater reacts with wetting agents in the film to allow the film to spread evenly over the entire surface of the shingle or tile.

An attempt to prevent the loss of algaecide from an algaecide-coated roofing shingle has been demonstrated in U.S. Pat. No. 3,932,143. The exposed portion of a roofing shingle is coated with copper and then further coated with an acrylic coating in its entirety. While this may extend the life of the shingle, it does not allow rainwater to contact the algaecide and permit it to spread evenly over the surface of the shingle.

Another method of controlling algae growth is to coat roofing granules with algaecide and apply them to the exposed surface of a shingle. U.S. Pat. No. 3,894,877 incorporates copper silicate with a heavy processing oil, the mixture of which is used to coat color coated roofing granules. The copper silicate adheres to the surface of the granule color coating.

U.S. Pat. No. 3,888,684 illustrates coating roofing granules with copper and zinc ionizable algaecides. The granules have an inner and outer coating; the inner coating having and algaecidal content of twice the algaecidal content of the outer coating.

U.S. Pat. No. 5,427,793 discloses coating roofing granules with a tin-acrylate polymer oil coating. U.S. Pat. No. 5,573,782 illustrates a composite sheet with a granular surface material. The entire exposed surface of the sheet is coated with a tin-acrylate polymer oil coating.

U.S. Pat. No. 5,573,810 shows a method applying microorganism-resistant granules to an asphalt sheet.

All of the past methods of preventing algae growth on shingles and siding have limited durability or are limited in their effectiveness. Algaecide applied on the exposed surface of a shingle or siding is gradually washed away by moisture causing the surface to lose its capability of inhibiting algae growth. Applying a coating over the algaecide coated shingle or siding will prevent the algaecide from washing away, but in turn, will not allow for the even distribution of the algaecide on the entire surface of the siding or shingle and thereby limit its effectiveness.

Another problem with past methods is that it is very costly to coat an entire surface of a shingle or siding panel, especially with coatings containing copper or zinc metal compounds. Further, coating the entire surface of a shingle or siding panel, particularly with copper and/or zinc, leads to a higher amount of algaecide runoff potentially contributing to increased levels of the coating materials in groundwater and/or stormwater.

### SUMMARY OF THE INVENTION

Accordingly, it is an object of the present invention to provide an algae-resistant exterior panel in which less algaecide is washed away by rainwater, melting frost, dew and other forms of moisture.

It is another object of the present invention to provide an algae-resistant exterior panel in which the algaecide provides algae control without coating the entire exterior surface of the panel with algaecide.

It is an object of the present invention to provide an algae-resistant exterior panel in which only a small portion of the panel is covered with algaecide.

It is another object of the present invention to provide an algae-resistant exterior panel which requires less algaecide and is less expensive to produce.

It is an object of the present invention to provide an algae-resistant exterior panel, which provides longer periods of algae inhibition.

It is a further object of the present invention to provide an algae-resistant exterior panel which contributes less potential runoff.

According to the present invention, an exterior panel containing algaecidal properties is provided for inhibiting the growth of algae on roofing materials, and exterior siding. Algaecide is applied to the exterior panel on the unexposed surface, i.e., the side of the panel, when installed, which is not directly in contact with the effects of weather. When moisture such as rain, dew, melting frost or snow, contacts the exposed surface of the panel, water is transported to the unexposed surface of the panel via capillary attraction. The water extracts the algaecide and distributes it to the exposed surfaces of the panels and all of the other descending panels.

The foregoing and other advantages of the invention will become apparent from the following disclosure in which one or more preferred embodiments of the invention are described in detail and illustrated in the accompanying drawings. It is contemplated that variations in procedures, structural features and arrangement of parts may appear to a person skilled in the art without departing from the scope of or sacrificing any of the advantages of the invention.

### BRIEF DESCRIPTION OF THE DRAWINGS

FIG. 1 is a side view of an exterior panel according to the present invention.

FIG. 2 is a side view of a preferred embodiment of the present invention.

FIG. 3 is a side view of an alternative embodiment of the present invention.

In describing preferred embodiments of the invention which are illustrated in the drawings, specific terminology is resorted to for the sake of clarity. However, it is not intended that the invention be limited to the specific terms so selected and it is to be understood that each specific term includes all technical equivalents that operate in a similar manner to accomplish a similar purpose.

Although preferred embodiments of the invention are herein described, it is understood that various changes and modifications in the illustrated and described structure can be affected without departure from the basic principles that underlie the invention. Changes and modifications of this type are therefore deemed to be circumscribed by the spirit and scope of the invention, except as the same may be necessarily modified by the appended claims or reasonable equivalents thereof.

### DETAILED DESCRIPTION AND PREFERRED EMBODIMENTS OF THE INVENTION

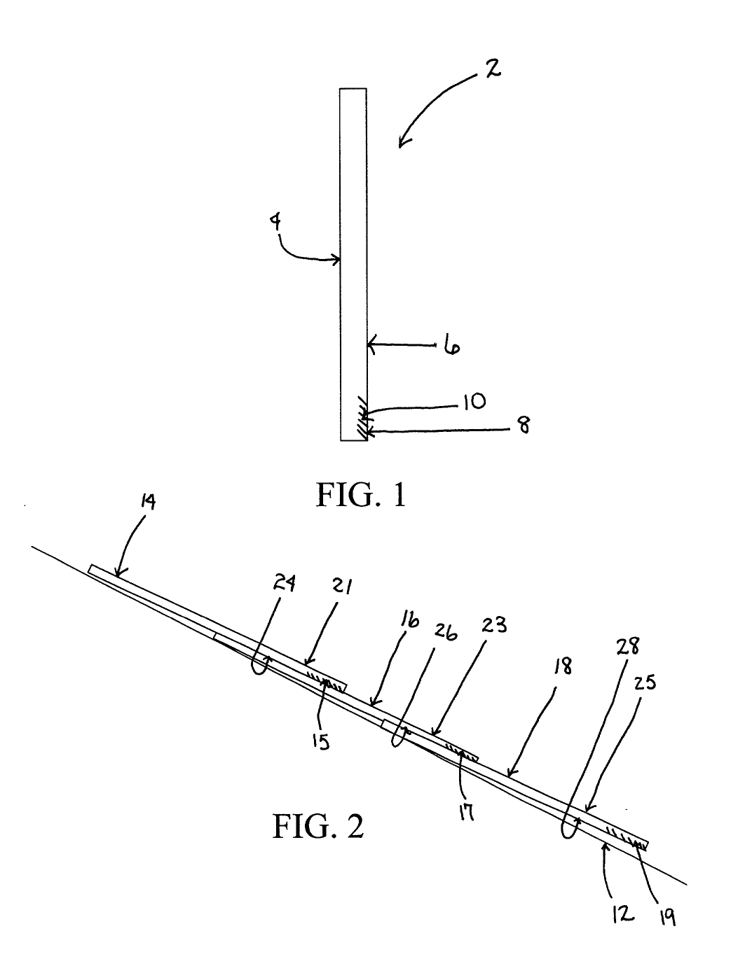

Referring now to the drawings, in FIG. 1, the illustrated exterior panel 2 includes an exposed surface 4 and an unexposed surface 6. The exposed surface 4 of the panel is the surface, when installed on the exterior of a building, such as upon a roof or side of the building, which is normally visible and/or comes into contact with the outside environment. The exposed surface 4 is exposed to the weathering effects of rain, dew, melting frost, and other moisture. The exterior panel of the present invention may be a roofing product, such as an asphalt shingle, clay tile, cement tile, a cedar shake, strip shingles, rolled roofing, a glass fiber reinforced polymeric panel; or may be any other exterior panel, such as vinyl siding, cement siding, cedar siding, aluminum siding or an exterior accessory panel.

Algaecide 10 is applied to unexposed surface 6 of the panel. Preferably, the algaecide is applied on the lower 12 to 51 mm of the butt end 8 of the exterior panel 2. The algaecide may be any commonly used algae-inhibiting agent including copper, copper compounds, zinc, zinc compounds, tin, tin compounds, silver-containing zeolites, organic biocides and combinations thereof. The algaecide may be in the form of coated roofing granules, paints, powders, organic coatings or adhesive coatings which are chosen to provide water permeability to promote the extraction of the algaecide or an ion exchange therewith, through contact with moisture. Alternatively, adhesive strips containing algaecide can be adhered to the unexposed surface 6 of the exterior panel.

Preferably, algaecidal roofing granules are applied to the unexposed surface of the exterior panel. Algaecidal granules are applied in an amount effective to inhibit algae growth, the amount varying with the type of algaecide being used. One skilled in the art appreciates that an effective amount of algaecide prevents significant amounts of algae growth for a prolonged period, preferably several years. Typical granules used in the art are described in detail in U.S. Pat. No. 5,536,664, 5,411,803 and 5,382,475 herein incorporated by reference in their entirety.

In a preferred embodiment of the present invention, copper oxide coated granules are used as algaecide. The copper is applied by the granule manufacturer and fired along with the pigments in the color coating of the granules by standard granule manufacturing methods. Applicants have found in a limited number of tests that an effective amount of copper oxide coated granules includes the range of about 0.5 to 2.5 grams per kilogram substrate granules applied to an asphalt roofing shingle according to the present invention.

The coated granules are then applied to the unexposed side of asphalt roofing shingles during the manufacture of the shingles. The basic shingle is manufactured in a known manner, such as described in commonly assigned U.S. Pat. No. 6,014,847, and therefore not described in detail herein. Typically, the asphalt shingle substrate is first coated with a mixture of hot asphalt and fillers such as limestone, sand or stone dust. The coated granules are then applied to the back of the substrate, directly on the hot filled coating, using the asphalt to adhere the granules in place. Preferably, the granules are applied on the lower 12 to 51 mm of the butt end 8.

These copper oxide coated granules are ionizable upon contact with moisture permeating the granule coatings during periods of rain, dew or melting snow or ice. The ionization of the algaecidal compounds and the resulting slow release and leaching of the algaecidal ions for passage over the exterior panel surface produces the desired effect of inhibiting algae growth on the surface of the exterior panel. As used herein, extraction of algaecide may refer to the leaching of ions or the transport of the algaecide material.

FIG. 2 depicts asphalt roofing shingles 14, 16 and 18 as installed on roof 12. The roofing shingles are treated with an algaecide according to the present invention. Such shingles can be asphalt shingles, clay tiles, Spanish tiles, a reinforced glass fiber polymeric shingle or any other roofing material such as cedar shakes, rolled roofing material, thermoformed or molded plastic roofing. The roofing shingles abut one another, the algaecide-treated unexposed surfaces 24, 26 and 28 coming into contact with the exposed surfaces 21 and 23.

The algaecide 15, 17 and 19 is adhered to the unexposed surfaces 24, 26 and 28 of the roofing shingles. When rain, dew or other moisture contacts the exposed surfaces 21 and 23 of the shingles the moisture is transported to the unexposed surfaces 24, 26 and 28 by capillary action between the shingles. The water carries the algaecide to the exposed surfaces 21, 23 and the algaecide is distributed over the exposed surfaces 21 and 23 of the shingles and all of the other descending shingles (not shown). As such, the algaecide is transported by the moisture from the unexposed surfaces to the exposed surfaces to protect the exposed surfaces from algae growth.

In another embodiment, silver-containing zeolites are used as the algaecide. The zeolites are in the form of a dry powder such as that manufactured by Health Shield, Westport, Conn. The powder is applied onto the surface of the hot asphalt coating as described above. The powder may be applied with or without the coated granules. The silver-containing zeolites may also be formulated into an organic coating or paint that is sprayed or roll-coated onto the top of the substrate and dried by air or heat. The silver-containing zeolites may be applied to the exterior panel in the amount of 0.01 to 1.0 grams per square foot, preferably 0.1 grams per square foot.

Alternatively, algaecides may be applied as a film, or as an adhesive or in the form of an algaecidal tape on the unexposed surface of the exterior panel. In addition, algaecides may be sprayed or brushed on an exterior panel as a metal coating, film or paint. These adhesives, coatings, films, and paints are chosen to provide water permeability to promote the extraction of algaecide in the manner described above.

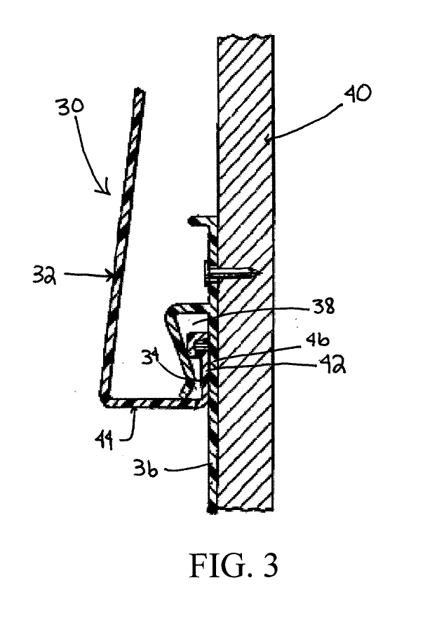

Referring now to FIG. 3, an alternative embodiment of the present invention is shown in which exterior siding is treated with an algaecide. FIG. 3 depicts typical siding panels as installed on the outside of a building. First panel 30 includes an exterior exposed surface 32 an upwardly directed lip 34 on the bottom edge of the panel which projects into and interlocks with a channel 38 on a second panel 36 mounted below the first panel 30 on a building wall 40. Algaecide (not shown) is applied to unexposed edge 42 of the lip 34 in a manner similar to that disclosed with reference to FIGS. 1 and 2. Alternatively, algaecide can be applied to unexposed surface 46 of second panel 36 in the region adjacent to the unexposed edge 42 of panel 30.

When rain or other moisture comes into contact with the exterior exposed surface 32 of first panel 30, the water is transported along the exterior exposed surface 32 to horizontal edge 44 and to the back unexposed edge 42 of the lip 34 of the first panel 30. As the water extracts algaecide from the edge 42, the algaecide is then distributed over second panel 36.

Algaecides may be provided in the form of adhesives, films or coatings, as described above, and may be rolled or sprayed on during or after the manufacturing process. Alternatively, an algaecidal adhesive tape may also be applied.

It is possible that changes in configurations to other than those shown could be used but that which is shown is preferred and typical. It is therefore understood that although the present invention has been specifically disclosed with the preferred embodiment and examples, modifications to the design concerning sizing and shape will be apparent to those skilled in the art and such modifications and variations are considered to be equivalent to and within the scope of the disclosed invention and the appended claims.

What is claimed is:

1. An exterior panel comprising:
a) an exposed surface;
b) an unexposed surface; and
c) an effective amount of algaecide provided on said unexposed surface to inhibit algae growth on a second exposed surface provided on a second adjacent panel.

2. The exterior panel of claim 1, wherein said exterior panel comprises a butt end, wherein said algaecide is provided on said unexposed surface of said butt end.

3. The exterior panel claim 2, wherein said algaecide is provided on the lower 12 mm to 51 mm of the butt end.

4. The exterior panel of claim 1, wherein the unexposed surface is installed adjacent to the exposed surface of the second exterior panel.

5. The exterior panel of claim 1, wherein said algaecide is selected from the group consisting of copper, copper compounds, zinc, zinc compounds, chromium, chromium compounds, tin compounds, organic biocides, silver-containing zeolites and combinations thereof.

6. The exterior panel of claim 1, wherein said algaecide is applied in an amount of about 0.1 to about 2.5 gram per square foot.

7. The exterior panel of claim 6, wherein said algaecide is applied in an amount of about 0.5 to about 2.5 gram per square foot.

8. The exterior panel of claim 7, wherein said algaecide is applied in an amount of about 0.1 to about 1.0 gram per square foot.

9. The exterior panel of claim 1, wherein said algaecide comprises silver-containing zeolites.

10. The exterior panel of claim 9, wherein said algaecide is applied in an effective amount to inhibit algae growth.

11. The exterior panel of claim 9, wherein said algaecide comprises a powder.

12. The exterior panel of claim 11, wherein said algaecide is applied in an amount of about 0.1 to 1.0 gram per square foot.

13. The exterior panel of claim 12, wherein said algaecide is applied in an amount of about 0.1 gram per square foot.

14. The exterior panel of claim 1, wherein said exterior panel is a roofing product.

15. The exterior panel of 14, wherein said exterior panel is an asphalt shingle.

16. The exterior panel of claim 1, wherein said exterior panel is a clay tile.

17. The exterior panel of claim 1, wherein said exterior panel is manufactured from a cementitious material.

18. The exterior panel of claim 1, wherein said exterior panel is a glass fiber reinforced polymeric member.

19. The exterior panel of claim 1, wherein said exterior panel is siding.

20. The exterior panel of claim 1, further comprising said exposed surface having no substantial amount of algaecide provided thereon.

21. A process of applying algaecide to an exterior panel comprising the steps of:
a) providing a first exterior panel with an exposed side and an unexposed side;
b) applying an algaecide to said unexposed side in an amount effective to prevent algae growth on an exposed side of a second adjacent panel.

22. The process of claim 21, wherein no significant amount of said algaecide is applied to said exposed side of said first exterior panel.

23. The process of claim 21, wherein said algaecide is applied by spraying said algaecide on said exterior panel.

24. The process of claim 21, wherein said algaecide is applied by providing said algaecide in a paint and applying said paint to said exterior panel.

25. The process of claim 21, wherein said algaecide is applied by adhering an algaecidal film on said exterior panel.

26. The process of claim 21, wherein said algaecide is selected from the group consisting of copper, copper compounds, zinc, zinc compounds, chromium, chromium compounds, tin compounds, organic biocides, silver-containing zeolites and combinations thereof.

27. A method for preventing algae growth on exterior panels comprising the steps of:
a) providing a first exterior panel with an exposed and an unexposed surface;
b) providing a second exterior panel with an exposed and unexposed surface;
c) providing algaecide on the unexposed surface of said second exterior panel;
d) securing said first exterior panel to a building structure;
e) securing a second exterior panel adjacent to said first exterior panel so that the unexposed surface of said second exterior panel is in an abutting position to said exposed surface of said first exterior panel; and
f) providing moisture on said exterior panels, said moisture transporting said algaecide from the unexposed surface of said second exterior panel to said exposed surface of said first exterior panel.

28. The method of claim 27, wherein said algaecide is selected from the group consisting of copper, copper compounds, zinc, zinc compounds, chromium, chromium compounds, tin compounds, organic biocides, silver-containing zeolites and combinations thereof.

29. The method of claim 28, wherein said algaecide provided on the unexposed surface is applied in an amount effective to inhibit algae growth on said exposed surface of said first panel.

30. The method of claim 28, wherein said second exterior panel comprises a butt end, wherein said algaecide is provided on said unexposed surface of said butt end.

31. The method of claim 30, wherein said algaecide is provided on the lower 12 mm to 51 mm of the butt end.

32. The method of claim 27, wherein said moisture comprises water.

33. The exterior panel claim 27, wherein said first exterior panel and said second exterior panel are asphalt shingles.

34. The exterior panel of claim 27, wherein said first exterior panel and said second exterior panel are clay tiles.

35. The exterior panel of claim 27, wherein said first exterior panel and said second exterior panel are cement members.

36. The exterior panel of claim 27, wherein said first exterior panel and said second exterior panel are glass fiber reinforced polymeric members.

37. The method of making an algae-resistant shingle comprising the steps of:
a) providing an asphaltic strip with an exposed surface and an unexposed surface;
b) providing a tacky surface on said unexposed surface; and
c) adhering particles with algaecidal properties to said unexposed surface.

38. The method of claim 37, wherein said tacky surface is liquid asphalt.

39. The method of claim 37, wherein said particles with algaecidal properties comprise algaecide selected from the group consisting of copper, copper compounds, zinc, zinc compounds, chromium, chromium compounds, tin compounds, organic biocides, silver-containing zeolites and combinations thereof.

40. The method of claim 37, wherein said algaecide provided on the unexposed surface is applied in an amount effective to inhibit algae growth on said exposed surface of said first panel.

41. The method of claim 37, wherein said strip is an asphalt shingle.
